(19) United States
(12) Patent Application Publication
(10) Pub. No.: US 2002/0102242 A1
(43) Pub. Date: Aug. 1, 2002

(54) Compositions and methods for administering pneumococcal DNA

(76) Inventors: David E. Briles, Birmingham, AL (US); Larry S. McDaniel, Ridgland, MS (US); David T. Curiel, Birmingham, AL (US)

Correspondence Address: William S. Frommer, FROMMER LAWRENCE & HAUG LLP, 745 Fifth Avenue, New York, NY, 10151, US

(21) Appl. No.: 09/844,645

(22) Filed: Apr. 27, 2001

Related U.S. Application Data
(63) Continuation of application No. 08/759,505, filed on Dec. 4, 1996.

(51) Int. Cl. A61K 48/00; A61K 39/02; C12N 15/86

(52) U.S. Cl. 424/93.21; 424/190.1; 435/320.1; 435/456

(57) ABSTRACT

Plasmid DNA encoding at least one pneumococcal antigen or epitope of interest and methods for making and using such a plasmid are disclosed and claimed. The epitope of interest can be PspA or a fragment thereof. Compositions containing the plasmid DNA are useful for administration to a host susceptible to pneumococcal infection for an in vivo response, such as a protective response, or for generating useful antibodies. The inventive plasmid can also be transfected into cells for generating antigens or epitopes of interest in vitro. And the inventive plasmid can be prepared by isolating DNA (coding for: promoter, leader sequence, epitope of interest and terminator), and performing a three-way ligation. More particularly, administration of DNA encoding pneumococcal antigens or epitopes of interest and compositions therefor for eliciting and immunological response against S. pneumoniae, such as a protective response preventive of pneumococcal infection, are disclosed and claimed. Thus, pneumococcal vaccines or immunological compositions, and methods of making and using them, are disclosed and claimed.

### FIELD OF THE INVENTION

This invention relates to compositions and methods for administering pneumococcal DNA encoding antigen(s) or epitopes of interest thereof in vivo, ex vivo or in vitro. More particularly, this invention relates to compositions and methods for administering pneumococcal DNA encoding an antigen(s) or epitopes of interest, e.g., PspA (pneumococcal surface protein A) or fragments thereof, for expression thereof, in vivo, ex vivo or in vitro.

### BACKGROUND OF THE INVENTION

Streptococcus pneumoniae is an important cause of otitis media, meningitis, bacteremia and pneumonia. Despite the use of antibiotics and vaccines, the prevalence of pneumococcal infections has declined little over the last twenty-five years.

It is generally accepted that immunity to Streptococcus pneumoniae can be mediated by specific antibodies against the polysaccharide capsule of the pneumococcus. However, neonates and young children fail to make an immune response against polysaccharide antigens and can have repeated infections involving the same capsular serotype.

One approach to immunizing infants against a number of encapsulated bacteria is to conjugate the capsular polysaccharide antigens to protein to make them immunogenic. This approach has been successful, for example, with Haemophilus influenzae b (see U.S. Pat. No. 4,496,538 to Gordon and U.S. Pat. No. 4,673,574 to Anderson). However, there are over eighty known capsular serotypes of S. pneumoniae of which twenty-three account for most of the disease. For a pneumococcal polysaccharide-protein conjugate to be successful, the capsular types responsible for most pneumococcal infections would have to be made adequately immunogenic. This approach may be difficult, because the twenty-three polysaccharides included in the presently-available vaccine are not all adequately immunogenic, even in adults.

An alternative approach for protecting children, and also the elderly, from pneumococcal infection would be to identify protein antigens that could elicit protective immune responses. Such proteins may serve as a vaccine by themselves, may be used in conjunction with successful polysaccharide-protein conjugates, or as carriers for polysaccharides.

McDaniel et al. (I), J. Exp. Med. 160:386-397, 1984, relates to the production of hybridoma antibodies that recognize cell surface polypeptide(s) on S. pneumoniae and protection of mice from infection with certain strains of encapsulated pneumococci by such antibodies.

This surface protein antigen has been termed “pneumococcal surface protein A”, or “PspA” for short.

McDaniel et al. (II), Microbial Pathogenesis 1:519-531, 1986, relates to studies on the characterization of the PspA. Considerable diversity in the PspA molecule in different strains was found, as were differences in the epitopes recognized by different antibodies.

McDaniel et al. (III), J. Exp. Med. 165:381-394, 1987, relates to immunization of X-linked immunodeficient (XID) mice with non-encapsulated pneumococci expressing PspA, but not isogenic pneumococci lacking PspA, protects mice from subsequent fatal infection with pneumococci.

McDaniel et al. (IV), Infect. Immun., 59:222-228, 1991, relates to immunization of mice with a recombinant full length fragment of PspA that is able to elicit protection against pneumococcal strains of capsular types 6A and 3.

Crain et al, Infect.Immun., 56:3293-3299, 1990, relates to a rabbit antiserum that detects PspA in 100% (n=95) of clinical and laboratory isolates of S. pneumoniae. When reacted with seven monoclonal antibodies to PspA, fifty-seven S. pneumoniae isolates exhibited thirty-one different patterns of reactivity.

Above cited applications Ser. No. 08/529,055, filed Sep. 15, 1995, Ser. No. 08/470,626, filed Jun. 6, 1995, Ser. No. 08/467,852, filed Jun. 6, 1995, Ser. No. 08/469,434, filed Jun. 6, 1995, Ser. No. 08/468,718, filed Jun. 6, 1995, Ser. No. 08/247,491, filed May 23, 1994, Ser. No. 08/214,222, filed Mar. 17, 1994 and Ser. No. 08/214,164, filed Mar. 17, 1994, Ser. No. 08/246,636, filed May 20, 1994, and Ser. No.08/319,795, filed Oct. 7, 1994, and U.S. Pat. No. 5,476,929, relate to vaccines comprising PspA and fragments thereof, methods for expressing DNA encoding PspA and fragments thereof, DNA encoding PspA and fragments thereof, the amino acid sequences of PspA and fragments thereof, compositions containing PspA and fragments thereof and methods of using such compositions.

The PspA protein type is independent of capsular type. It would seem that genetic mutation or exchange in the environment has allowed for the development of a large pool of strains which are highly diverse with respect to capsule, PspA, and possibly other molecules with variable structures. Variability of PspA's from different strains also is evident in their molecular weights, which range from 67 to 99 kD. The observed differences are stably inherited and are not the result of protein degradation.

Immunization with a partially purified PspA from a recombinant λgt11 clone, elicited protection against challenge with several S. pneumoniae strains representing different capsular and PspA types, as in McDaniel et al. (IV), Infect. Immun. 59:222-228, 1991. Although clones expressing PspA were constructed according to that paper, the product was insoluble and isolation from cell fragments following lysis was not possible.

While the protein is variable in structure between different pneumococcal strains, numerous cross-reactions exist between all PspA's, suggesting that sufficient common epitopes may be present to allow a single PspA or at least a small number of PspA's to elicit protection against a large number of S. pneumoniae strains.

In addition to the published literature specifically referred to above, the inventors, in conjunction with co-workers, have published further details concerning PspA's, as follows:

1. Abstracts of 89th Annual Meeting of the American Society for Microbiology, p. 125, item D-257, May 1989;

2. Abstracts of 90th Annual Meeting of the American Society for Microbiology, p. 98, item D-106, May 1990;

3. Abstracts of 3rd International ASM Conference on Streptococcal Genetics, p. 11, item 12, June 1990;

4. Talkington et al, Infect. Immun. 59:1285-1289, 1991;

5. Yother et al (I), J. Bacteriol. 174:601-609, 1992; and

b 6. Yother et al (II), J. Bacteriol. 174:610-618, 1992.

7. McDaniel et al (V), Microbiol. Pathogenesis, 13:261-268.

Alternative vaccination strategies are desirable as such provide alternative routes to administration or alternative routes to responses.

In particular, it is believed that heretofore the art has not taught or suggested administration to a eukaryotic cell in vitro or ex vivo, or to a mammalian host—domesticated, wild or human—susceptible to pneumococcal infection, DNA encoding PspA and/or fragments thereof (including epitopes of interest), or expression thereof in vivo, especially as herein disclosed.

### OBJECTS AND SUMMARY OF THE INVENTION

It is an object of the invention to provide methods and compositions for administering to a host, such as a mammalian host, including human, susceptible to pneumococcal infection, isolated and/or purified pneumococcal DNA encoding an epitope of interest, such as an antigen or antigens, e.g., isolated and/or purified DNA encoding a PspA or a fragment thereof or a combination thereof. The compositions can include a carrier or diluent. The DNA is administered in a form to be expressed by the host, i.e., such that there is an expression product of the DNA, and preferably in an amount sufficient to induce a response such as a protective immune response; and, the DNA can be administered without any necessity of adding any immunogenicity-enhancing adjuvant.

Accordingly, the present invention provides pneumococcal epitopes of interest, DNA plasmids for expression of an expression product by eukaryotic cells, compositions containing the plasmids, and methods for using the compositions and for using the products from the compositions.

The plasmid of the invention can comprise, from upstream to downstream (5′ to 3′): DNA encoding a promoter for driving expression in a eukaryotic cell, DNA encoding a leader sequence which facilitates expression, and also preferably, translation through or transport of the expression product in a eukaryotic cell membrane and DNA encoding a pneumococcal antigen or epitope of interest. The plasmid can optionally contain additional DNA for regulating expression, such as DNA encoding at least one enhancer, terminator, etc. The eukaryotic cell is preferably a mammalian cell.

Moreover, the invention provides an immunological composition comprising the aforementioned plasmid and a suitable carrier or diluent, as well as a method for eliciting an immunological response in a host susceptible to pneumococcal infection or sepsis, comprising the administration of said immunological composition.

Further, the invention provides a vaccine comprising the aforementioned plasmid and a suitable carrier or diluent, and optionally one or more cytokines or DNA encoding the same, or a bacterial delivery system. If instead of a cytokine, DNA encoding a cytokine is present, such DNA can be within the inventive plasmid, either upstream or downstream from the pneumococcal DNA, or in a plasmid of its own. The cytokine DNA plasmid can comprise, from upstream to downstream (5′ to 3′): DNA encoding a promoter for driving expression in a eukaryotic cell, DNA encoding a leader sequence for facilitating expression in a eukaryotic cell, and also preferably transport through the eukaryotic cell membrane, and DNA encoding a cytokine or epitope of interest thereof. The DNA encoding a cytokine or epitope of interest thereof can be as in U.S. Pat. No. 5,252,479 and WO 94/16716, which provide genes for cytokines and tumor associated antigens and immunotherapy methods, including ex vivo methods, incorporated herein by reference. This cytokine plasmid can also contain additional DNA for regulating expression (e.g., encoding at least one enhancer, a terminator, etc.); and the eukaryotic cell is preferably a mammalian cell.

An epitope of interest is an antigen or immunogen or immunologically active fragment thereof from a pathogen or toxin of veterinary or human interest.

The invention additionally provides a plasmid comprising DNA encoding a promoter, DNA encoding a leader sequence to facilitate translation of the expression product of the plasmid through a mammalian cell membrane, and DNA encoding a pneumococcal epitope of interest wherein the DNA encoding the leader sequence encodes a protein which facilitates translation of the expression product through the mammalian cell membrane, and adhesion thereto, by being expressed with the pneumococcal DNA as a fusion protein.

These and other embodiments are disclosed or are obvious from the following detailed description.

### BRIEF DESCRIPTION OF THE FIGURES

FIG. 1A shows pGT41, constructed using the commercially available pcDNA3;

FIG. 1B shows the sequence of pcDNA3;

FIG. 1C shows the sequence of rsvG which was amplified, digested with KpnI and ligated into pcDNA3;

FIG. 1D shows the construction of pKSD2601 (to construct the PspA+ plasmid, the amplified fragment encoding PspA was inserted as a BamHI-EcoRI fragment into the expression vector pGT41, and the resulting rsvG::pspA is under control of the CMV (cytomegalovirus) promoter); and

FIG. 2 shows the survival of BALB/c mice challenged with capsular type 3 S. pneumoniae A66 (groups of five mice, in two different experiments for a total of ten mice per curve, were immunized with the vector, pGT41, only, as a control, or with the PspA+vector, pKSD2601, and all mice were challenged with 100×LD₅₀ of A66 intravenously).

### DETAILED DESCRIPTION

Knowledge of and familiarity with the applications incorporated herein by reference is assumed; and, those applications disclose the sequence of pspA as well as certain portions thereof including portions thereof containing epitopes, and PspA and compositions containing PspA.

Direct injection of plasmid DNA has become a simple and effective method of vaccination against a variety of infectious diseases (see, e.g., Science, 259: 1745-49, 1993). Since the first demonstration of the ability of naked plasmid DNA to elicit protective immune responses, DNA immunization has been shown to be an effective means of eliciting immunity in a number of model systems (see, e.g., Vaccine, 12: 1541-44, 1994). It is potentially more potent and longer lasting than recombinant protein vaccination because it elicits both humoral as well as a cellular immune response.

The present invention provides a DNA-based vaccine or immunological composition against pneumococcal infection, and can elicit an immunological response, which can confer protection in mice against challenge with an infectious strain of Streptococcus pneumoniae (and ergo in other mammalian hosts susceptible thereto, such as humans). An exemplary plasmid of the invention contains the human cytomegalovirus immediate early (HCMV-IE) promoter driving expression of full-length PspA, and a portion of the gene which encodes RSVG (respiratory syncytial virus glycoprotein G), such that when an in-frame fusion is made, the resultant fusion protein is transported to, and anchored in, the mammalian cell membrane, where it is exposed to the host immune system. As to the HCMV-IE promoter, reference is made to U.S. Pat. Nos. 5,168,062 and 5,385,839, incorporated herein by reference.

Expression and secretion was demonstrated in cultured HeLa cells, and the transfected cells were stained for both cytoplasmic and surface expression of PspA using anti-PspA monoclonal antibodies (MAbs) and a fluorescently labeled secondary antibody, as previously described in McDaniel, L.S., et al., Infect. Immun. 56: 3001-3003, 1988. PspA was expressed in the cytoplasm of the HeLa cells, but it was not present on the surface of the cells. In addition, one of two MAbs, XiR278, detected PspA.

Protection was demonstrated in BALB/c mice by lingual injection of naked plasmid DNA, and subsequently challenging with capsular serotype 3 S. pneumoniae A66. It was found that the immunized mice had approximately 1.5 to 2 times less pneumococci per ml of blood (on a logarithmic scale) than the control mice which received the vector alone with no pneumococcal DNA inserted (see Table 2).

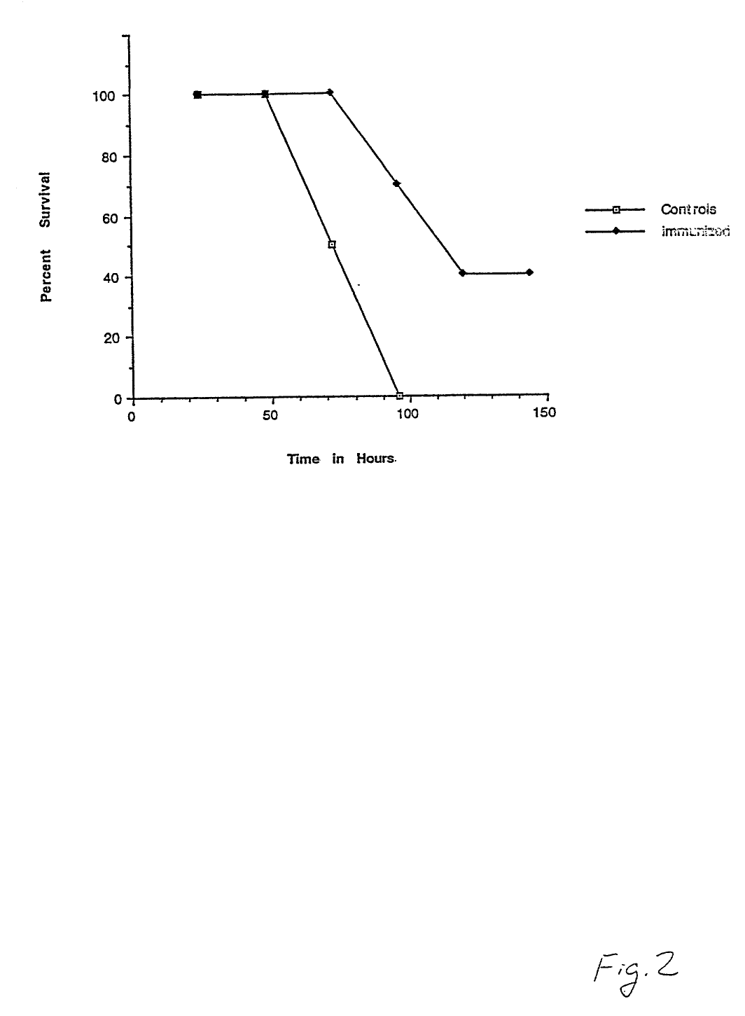

Moreover, the effects of immunization could also be seen in protection from death (see FIG. 2). Forty percent of the mice that were immunized with naked plasmid DNA survived challenge, whereas none of the control mice survived. In addition, the median time of death of the mice immunized with naked plasmid DNA was approximately 100 hours, as compared to that of 75 hours for the control group. Hence, intramuscular immunization with naked plasmid DNA can induce protection against an otherwise lethal challenge with a capsular type 3 pneumococcus.

Thus, a DNA vaccine or immunological composition expressing pneumococcal epitope of interest, for instance, full-length PspA or a fragment thereof or combinations thereof, can protect mice, and ergo other mammals such as humans, against infection by the etiologic agent of pneumococcal infection. The composition is thus useful for eliciting a protective response in a host susceptible to pneumococcal infection, as well as for eliciting antigens and antibodies, which also are useful in and of themselves.

Therefore, as discussed above, the invention in a general sense, preferably provides methods for immunizing, or vaccinating, or eliciting an immunological response in a host, such as a host susceptible to pneumococcal infection, e.g., a mammalian host, by administering DNA encoding a pneumococcal epitope of interest, for instance DNA encoding PspA or a fragment thereof or combinations thereof, in a suitable carrier or diluent, such as saline; and, the invention provides plasmids and compositions for performing the method, as well as methods for making the plasmids, and uses for the expression products of the plasmids, as well as for antibodies elicited thereby.

The present invention provides an immunogenic, immunological or vaccine composition containing the pneumococcal epitope of interest, DNA encoding the same or an expression product thereof, and a pharmaceutically acceptable carrier or diluent. An immunological composition containing the pneumococcal epitope of interest, DNA encoding the same or an expression product thereof, elicits an immunological response—local or systemic. The response can, but need not be, protective. Am immunogenic composition containing the pneumococcal epitope of interest, DNA encoding the same or an expression product thereof, likewise elicits a local or systemic immunological response which can, but need not be, protective. A vaccine composition elicits a local or systemic protective response. Accordingly, the terms “immunological composition” and “immunogenic composition” include a “vaccine composition” (as the two former terms can be protective compositions).

The invention therefore also provides a method of inducing an immunological response in a host mammal comprising administering to the host an immunogenic, immunological or vaccine composition comprising the pneumococcal epitope of interest, DNA encoding the same or an expression product thereof and a pharmaceutically acceptable carrier or diluent.

In the present invention, the DNA encoding a PspA epitope of interest, e.g., PspA, or a fragment thereof, can be administered in dosages and by techniques well known to those skilled in the medical or veterinary arts taking into consideration such factors as the age, sex, weight, species and condition of the particular patient, and the route of administration. The DNA encoding the PspA epitope of interest, e.g., pspA, or a fragment thereof, can be administered alone, or can be co-administered or sequentially administered with other epitopes or antigens, e.g., with other pneumococcal epitopes or antigens, or with DNA encoding other pneumococcal epitopes or antigens; and, the DNA encoding the PspA epitope of interest, e.g., PspA, or a fragment thereof, can be sequentially administered.

As broadly discussed above, the invention comprehends plasmids comprising DNA including pneumococcal antigen DNA for expression by eukaryotic cells. The DNA, from upstream to downstream (5′ to 3′), can comprise: DNA encoding a promoter for driving expression in eukaryotic cells, DNA encoding a leader sequence which is preferably DNA encoding a protein or portion thereof, e.g., DNA which enables transportation through and anchorage to the expression product (a resultant protein fusion) the eukaryotic cell membrane where it can be exposed to the host immune system or collected isolated and/or purified (if, for instance, expression in vitro) and DNA encoding a pneumococcal epitope of interest.

For instance, the promoter can be a eukaryotic viral promoter such as a herpes virus promoter, e.g., a human or murine cytomegalovirus promoter DNA. As to the murine cytomegalovirus promoter (mCMV), U.S. Pat. No. 4,963,481 to Stinski, directed to the mCMV immediate early (IE) promoter functionally linked to a heterologous transcription enhancer, U.S. Pat. No. 4,968,615 to Koszinowski, directed to mCMV IE enhancer and optionally promoter, and U.S. Pat. No. 4,963,481 to de Villiers, directed to mCMV IE promoter or promoting fragment linked to heterologous sequence are hereby incorporated herein by reference.

The DNA encoding a leader sequence can be any DNA suitable for facilitating expression, and preferably also transport through the cell membrane, of viral DNA in a eukaryotic cell, such as a mammalian cell. The leader sequence can encode a protein or portion thereof, such that when an in-frame fusion is made with the pneumococcal DNA, the resultant fusion protein may be transported through and anchored to the mammalian cell membrane. The leader sequence can thus be DNA encoding RSVG or a portion thereof. The DNA encoding a leader sequence is for facilitating secretion of a eukaryotic protein sequence from a mammalian cell and can be any suitable leader sequence.

The plasmid optionally can contain additional regulatory DNA, such as DNA for a terminator; for instance, the BGH terminator.

The DNA encoding the pneumococcal epitope of interest can be DNA which codes for full length PspA, or a fragment thereof. A sequence which codes for a fragment of PspA can encode that portion of PspA which contains an epitope of interest, such as a protection-eliciting epitope of the protein.

Regions of PspA have been identified from the Rx1 strain of S. pneumoniae which not only contain protection-eliciting epitopes, but are also sufficiently cross-reactive with other PspAs from other S. pneumoniae strains so as to be suitable candidates for the region of PspA to be incorporated into a plasmid or vaccine, immunological or immunogenic composition. Epitopic regions of PspA include residues 1 to 115, 1 to 314, 192 to 260 and 192 to 588. DNA encoding fragments of PspA can comprise DNA which codes for the aforementioned epitopic regions of PspA; or it can comprise DNA encoding overlapping fragments of PspA, e.g., fragment 192 to 588 includes 192 to 260, and fragment 1 to 314 includes 1 to 115 and 192 to 260. DNA encoding PspA, or a fragment thereof, can be inserted into a plasmid alone, or it can be inserted into a plasmid in tandum with DNA encoding other epitopes of interest, e.g., other pneumococcal epitopes of interest, or DNA encoding a cytokine. With respect to proteins which have homology to PspA or are like PspA (e.g., PspC) and DNA encoding the same, reference is made to copending U.S. applications Ser. No. 08/529,055, filed Sep. 15, 1995, U.S. Ser. No. _______ (Attorney Docket No. 454312-2460), filed Sep. 16, 1996, and U.S. Ser. No. 08/710,749, filed Sep. 20, 1996.

As to epitopes of interest, one skilled in the art can determine an epitope of immunodominant region of a peptide or polypeptide and ergo the coding DNA therefore from the knowledge of the amino acid and corresponding DNA sequences of the peptide or polypeptide, as well as from the nature of particular amino acids (e.g., size, charge, etc.) and the codon dictionary, without undue experimentation.

A general method for determining which portions of a protein to use in an immunological composition focuses on the size and sequence of the antigen of interest. “In general, large proteins, because they have more potential determinants are better antigens than small ones. The more foreign an antigen, that is the less similar to self configurations which induce tolerance, the more effective it is in provoking an immune response.” Ivan Roitt, Essential Immunology, 1988.

As to size, the skilled artisan can maximize the size of the protein encoded by the DNA sequence to be inserted into the viral vector (keeping in mind the packaging limitations of the vector). To minimize the DNA inserted while maximizing the size of the protein expressed, the DNA sequence can exclude introns (regions of a gene which are transcribed but which are subsequently excised from the primary RNA transcript).

At a minimum, the DNA sequence can code for a peptide at least 8 or 9 amino acids long. This is the minimum length that a peptide needs to be in order to stimulate a CD4+T cell response (which recognizes virus infected cells or cancerous cells). A minimum peptide length of 13 to 25 amino acids is useful to stimulate a CD8+ T cell response (which recognizes special antigen presenting cells which have engulfed the pathogen). See Kendrew, supra. However, as these are minimum lengths, these peptides are likely to generate an immunological response, i.e., an antibody or T cell response; but, for a protective response (as from a vaccine composition), a longer peptide is preferred.

With respect to the sequence, the DNA sequence preferably encodes at least regions of the peptide that generate an antibody response or a T cell response. One method to determine T and B cell epitopes involves epitope mapping. The protein of interest “is fragmented into overlapping peptides with proteolytic enzymes. The individual peptides are then tested for their ability to bind to an antibody elicited by the native protein or to induce T cell or B cell activation. This approach has been particularly useful in mapping T-cell epitopes since the T cell recognizes short linear peptides complexed with MHC molecules. The method is less effective for determining B-cell epitopes” since B cell epitopes are often not linear amino acid sequence but rather result from the tertiary structure of the folded three dimensional protein. Janis Kuby, Immunology, (1992) pp. 79-80.

Another method for determining an epitope of interest is to choose the regions of the protein that are hydrophilic. Hydrophilic residues are often on the surface of the protein and therefore often the regions of the protein which are accessible to the antibody. Janis Kuby, Immunology, (1992) P. 81.

Yet another method for determining an epitope of interest is to perform an X-ray cyrstallographic analysis of the antigen (full length)-antibody complex. Janis Kuby, Immunology, (1992) p. 80.

Still another method for choosing an epitope of interest which can generate a T cell response is to identify from the protein sequence potential HLA anchor binding motifs which are peptide sequences which are known to be likely to bind to the MHC molecule.

The peptide which is a putative epitope, to generate a T cell response, should be presented in a MHC complex. The peptide preferably contains appropriate anchor motifs for binding to the MHC molecules, and should bind with high enough affinity to generate an immune response. Factors which can be considered are: the HLA type of the patient (vertebrate, animal or human) expected to be immunized, the sequence of the protein, the presence of appropriate anchor motifs and the occurance of the peptide sequence in other vital cells.

An immune response is generated, in general, as follows: T cells recognize proteins only when the protein has been cleaved into smaller peptides and is presented in a complex called the “major histocompatability complex MHC” located on another cell's surface. There are two classes of MHC complexes—class I and class II, and each class is made up of many different alleles. Different patients have different types of MHC complex alleles; they are said to have a ‘different HLA type’.

Class I MHC complexes are found on virtually every cell and present peptides from proteins produced inside the cell. Thus, Class I MHC complexes are useful for killing cells which when infected by viruses or which have become cancerous and as the result of expression of an oncogene. T cells which have a protein called CD4 on their surface, bind to the MHC class I cells and secrete lymphokines. The lymphokines stimulate a response; cells arrive and kill the viral infected cell.

Class II MHC complexes are found only on antigen-presenting cells and are used to present peptides from circulating pathogens which have been endocytosed by the antigen-presenting cells. T cells which have a protein called CD8 bind to the MHC class II cells and kill the cell by exocytosis of lytic granules.

Some guidelines in determining whether a protein is an epitopes of interest which will stimulate a T cell response, include: Peptide length—the peptide should be at least 8 or 9 ammino acids long to fit into the MHC class I complex and at least 13-25 amino acids long to fit into a class II MHC complex. This length is a minimum for the peptide to bind to the MHC complex. It is preferred for the peptides to be longer than these lengths because cells may cut the expressed peptides. The peptide should contain an appropriate anchor motif which will enable it to bind to the various class I or class II molecules with high enough specificity to generate an immune response (See Bocchia, M. et al, Specific Binding of Leukemia Oncogene Fusion Protein Peptides to HLA Class I Molecules, Blood 85:2680-2684; Englehard, V H, Structure of peptides associated with class I and class II MHC molecules Ann. Rev. Immunol. 12:181 (1994)). This can be done, without undue experimentation, by comparing the sequence of the protein of interest with published structures of peptides associated with the MHC molecules. Protein epitopes recognized by T cell receptors are peptides generated by enzymatic degradation of the protein molecule and are prestnted on the cell surface in association with class I or class II MHC molecules.

Further, the skilled artisan can ascertain an epitope of interest by comparing the protein sequence with sequences listed in the protein data base. Regions of the protein which share little or no homology are better choices for being an epitope of that protein and are therefore useful in a vaccine or immunological composition. Regions which share great homology with widely found sequences present in vital cells should be avoided.

Even further, another method is simply to generate or express portions of a protein of interest, generate monoclonal antibodies to those portions of the protein of interest, and then ascertain whether those antibodies inhibit growth in vitro of the pathogen from which the from which the protein was derived. The skilled artisan can use the other guidelines set forth in this disclosure and in the art for generating or expressing portions of a protein of interest for analysis as to whether antibodies thereto inhibit growth in vitro. For example, the skilled artisan can generate portions of a protein of interest by: selecting 8 to 9 or 13 to 25 amino acid length portions of the protein, selecting hydrophylic regions, selecting portions shown to bind from X-ray data of the antigen (full length)-antibody complex, selecting regions which differ in sequence from other proteins, selecting potential HLA anchor binding motifs, or any combination of these methods or other methods known in the art.

Epitopes recognized by antibodies are expressed on the surface of a protein. To determine the regions of a protein most likely to stimulate an antibody response one skilled in the art can preferably perform an epitope map, using the general methods described above, or other mapping methods known in the art.

As can be seen from the foregoing, without undue experimentation, from this disclosure and the knowledge in the art, the skilled artisan can ascertain the amino acid and corresponding DNA sequence of an epitope of interest for obtaining a T cell, B cell and/or antibody response. In addition, reference is made to Gefter et al., U.S. Pat. No. 5,019,384, issued May 28, 1991, and the documents it cites, incorporated herein by reference (Note especially the “Relevant Literature” section of this patent, and column 13 of this patent which discloses that: “A large number of epitopes have been defined for a wide variety of organisms of interest. Of particular interest are those epitopes to which neutralizing antibodies are directed. Disclosures of such epitopes are in many of the references cited in the Relevant Literature section.”)

Further, the DNA encoding the pneumococcal epitope of interest can comprise more than one serologically complementary pspA molecule, so as to elicit better response, e.g., protection, for instance, against a variety of strains of pneumococci; and the invention provides a system of selecting PspAs for a multivalent composition which includes cross-protection evaluation so as to provide a maximally efficacious composition. A multivalent composition comprises selected epitopes encoded by different pspAs which would be cloned in tandem to make a broadly cross-protection eleciting vaccine. This would not, however, preclude fusing PspA to a different protein which would elicit a response against the second protein. In a preferred embodiment, important epitopes of multiple PspAs would be linked together to form a multivalent composition in order to opitimize cross-protection against pneumococcal infection. Note again, copending U.S. applications Ser. Nos. 08/529,055, filed Sep. 15, 1995, U.S. Ser. No. ______ (Attorney Docket No. 454312-2460), filed Sep. 16, 1996, and U.S. Ser. No. 08/710,749, filed Sep. 20, 1996.

The DNA in the present invention can comprise any suitable promoter or extraneous DNA sequences which would facilitate the expression of PspA in vivo or in vitro in a eukaryotic cell such as a mammalian cell, and for thus eliciting an immunological response to the expressed protein (if in vivo).

Further, the present invention provides a DNA molecule in which the pspA leader sequence is replaced by DNA sequences which permit the transport of PspA or an epitope of interest thereof through the eukaryotic (preferably mammalian) cell membrane. Additionally, the leader sequence may be substituted appropriately with a DNA sequence which would permit the transport of PspA through the cell membrane, followed by the deletion of the DNA encoding the C-terminal half of PspA, in an effort to maximize the secretion of an epitope of interest from the cell. The secretion of PspA in this manner maximizes B cell response, while if PspA were to remain in the cell, the T-cell responses are maximized; the latter are not necessarily which are not protective against pneumococcal infection. Accordingly, use of a particular leader sequence can cause expression of an epitope of interest from a plasmid containing DNA encoding more than that epitope of interest. In this aspect of the invention, a leader is chosed for its quality of cleaving at a particular motif of a protein, and the presence of that motif in PspA, downstream (N-terminal to C-terminal) from the epitope whose expression and transport is desired.

Moreover, the present invention provides a DNA molecule which comprises an appropriate leader sequence to facilitate transport across the cell membrane, as well as a membrane anchor to cause the surface expression of PspA. This type of modification should increase the ability of the antigen to elicit antibody responses.

The plasmid can be in admixture with any suitable carrier, diluent or excipient such as sterile water, physiological saline, and the like. Of course, the carrier, diluent or excipient should not disrupt or damage the plasmid DNA.

The compositions of the present invention, i.e., antigen, epitope of interest or plasmid encoding an antigen or epitope of interest can be administered in any suitable manner. The compositions can be in a formulation suitable for the manner of administration. The formulation can include: liquid preparations for orifice, e.g., oral, nasal, anal, vaginal, peroral, intragastric administration and the like, such as solutions, suspensions, syrups, elixirs; and liquid preparations for parenteral, subcutaneous, intradermal, intramuscular, intravenous administration, and the like, such as sterile solutions, suspensions or emulsions, e.g., for administration by injection. Mucosal administration, such as nasal, oral, genital, anal for local response, and/or parenteral, subcutaneous, intradermal or intramuscular administration for systemic response and compositions therefor, are presently preferred.

The plasmids of the invention can be used for in vitro expression of antigens or epitopes of interest by eukaryotic cells. Recovery of such antigens or epitopes can be by any suitable techniques; for instance, techniques analogous to the recovery techniques employed in the documents cited herein (such as the applications cited under Related Applications and the documents cited therein).

The thus expressed antigens or epitopes of interest can be used in immunological, antigenic or vaccine compositions, with or without an immunogenicity-enhancing adjuvant(“expressed antigen compositions”). Such compositions can be administered in dosages and by techniques well known to those skilled in the medical or veterinary arts taking into consideration such factors as age, sex, weight, species, condition of the particular patient, and the route of administration. These compositions can be administered alone or with other compositions, and can be sequentially administered.

Of course, for any composition to be administered to an animal of human, including the components thereof, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable immunological response, such as by titrations of sera and analysis thereof for antibodies or antigens, e.g., by ELISA and/or EFFIT analysis. Such determinations do not require undue experimentation from the knowledge of the skilled artisan, this disclosure and the documents cited herein. And, the time for sequential administeration can be ascertained without undue experimentation.

The route of administration for the expressed antigen or epitopic compositions can be oral, nasal, anal, vaginal, peroral, intragastric, parenteral, subcutaneous, intradermal, intramuscular, intravenous, and the like.

The expressed antigen or epitope of interest compositions can be solutions, suspensions, emulsions, syrups, elixers, capsules (including “gelcaps”—gelatin capsule containing a liquid antigen or fragment thereof preparation), tablets, hard-candy-like preparations, and the like. The expressed antigen compositions may contain a suitable carrier, diluent, or excipient such as sterile water, physiological saline, glucose or the like. The compositions can also be lyophilized. The compositions can contain auxiliary substances such as wetting or emulsifying agents, pH buffering agents, adjuvants, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Suitable dosages for plasmid compositions and for expressed antigen and epitope of interest compositions can also be based upon the examples below, and upon the documents herein cited. For example, suitable dosages can be 0.5-500 ug antigen or epitope of interest, preferably 0.5 to 50 ug antigen or epitope of interest, for instance, 1-10 ug antigen or epitope of interest in expressed antigen epitopic compositions. In plasmid compositions, the dosage should be a sufficient amount of plasmid to elicit a response analogous to the expressed antigen or epitopic compositions; or expression analogous to dosages in expressed antigen or epitopic compositions. For instance, suitable quantities of plasmid DNA in plasmid compositions can be 0.1 to 2 mg, preferably 1-10 ug.

Thus, in a broad sense, the invention further provides a method comprising administering a composition containing plasmid DNA including DNA encoding a pneumococcal antigen or antigens, or epitopes of interest: for expression of the antigen or antigens or epitopes of interest in vivo for eliciting an immunological, antigenic or vaccine (protective) response by a eukaryotic cell; or, for ex vivo or in vitro expression (i.e., the cell can be a cell of a host susceptible to pneumococcal infection, and the administering can be to a host susceptible to pneumococcal infection such as a mammal, e.g., a human; or, the cell can be an ex vivo or in vitro cell). The invention further provides a composition containing a pneumococcal antigen or antigens or epitope of interest from expression of the plasmid DNA by a eukaryotic cell, in vitro or ex vivo, and methods for administering such compositions to a host mammal susceptible to pneumococcal infection to elicit a response.

The invention provides a method of administering the DNA of the present invention in suitable admixture with cytokines, including any of IL-1 to IL-12, e.g., IL-1, IL-2, IL-4, IFNγ, D71 and TNFα, to enhance the immune response at the site of injection. In particular, IL-2 is a T-cell cytokine involved in TH2 B-cell antibody responses. TNGα and IFNγ induce non-immune cells to express MHC class II antigens, and thereby enable them to present antigens to T-cells. Further, DNA encoding these cytokines can be administered in suitable admixture with the DNA of the present invention.

If instead of a cytokine, DNA encoding a cytokine is present, such DNA can be within the inventive plasmid, either upstream or downstream from the pneumococcal DNA, or in a plasmid of its own. The cytokine DNA plasmid can comprise, from upstream to downstream (5′ to 3′): DNA encoding a promoter for driving expression in a eukaryotic cell, DNA encoding a leader sequence for facilitating expression in a eukaryotic cell, and also preferably transport through the eukaryotic cell membrane, and DNA encoding a cytokine or epitope of interest thereof. The DNA encoding a cytokine or epitope of interest thereof can be as in U.S. Pat. No. 5,252,479 or WO 94/16716, which provides genes for cytokines and tumor associated antigens and immunotherapy methods, including ex vivo methods, incorporated herein by reference. This cytokine plasmid can also contain additional DNA for regulating expression, and the eukaryotic cells is preferably a mammalian cell. Thus, the present invention provides a method of administering the DNA of the present invention in suitable admixture with cytokines, DNA encoding a cytokine within the inventive plasmid, either upstream or downstream from the pneumococcal DNA, or in a plasmid of its own, to enhance the immune response at the site of injection.

Further, the invention provides a method for administering the DNA of the present invention in a bacterial delivery system, such that bacteria carry the DNA of the present invention. Appropriate bacteria include Shigella flexneri and E.coli, and any bacteria having the ability to invade a host cell, and subsequently die and lyze after invasion, releasing the immunological DNA in the cytoplasm where it can be translated to express antigen or epitope of interest. Because the bacteria are destroyed in the process it would fail to cause disease.

Since the methods can stimulate an immune or immunological response, the inventive methods can be used for merely stimulating an immune response (as opposed to also being a protective response) because the resultant antibodies (without protection) are nonetheless useful. By eliciting antibodies, by techniques well-known in the art, monoclonal antibodies can be prepared and, those monoclonal antibodies, can be employed in well known antibody binding assays, diagnostic kits or tests to determine the presence or absence of pneumococcal antigens or to determine whether an immune response to the virus has simply been stimulated. Those monoclonal antibodies can also be employed in recovery or testing procedures, for instance, in immunoadsorption chromatography to recover or isolate a pneumococcal antigen or epitope of interest such as PspA or a fragment thereof.

Monoclonal antibodies are immunoglobulins produced by hybridoma cells. A monoclonal antibody reacts with a single antigenic determinant and provides greater specificity than a conventional, serum-derived antibody. Furthermore, screening a large number of monoclonal antibodies makes it possible to select an individual antibody with desired specificity, avidity and isotype. Hybridoma cell lines provide a constant, inexpensive source of chemically identical antibodies and preparations of such antibodies can be easily standardized. Methods for producing monoclonal antibodies are well known to those of ordinary skill in the art, e.g., Koprowski, H. et al., U.S. Pat. No. 4,196,265, issued Apr. 1, 1989, incorporated herein by reference.

Uses of monoclonal antibodies are known. One such use is in diagnostic methods, e.g., David, G. and Greene, H. U.S. Pat. No. 4,376,110, issued Mar. 8, 1983; incorporated herein by reference. Monoclonal antibodies have also been used to recover materials by immunoadsorption chromatography, e.g., Milstein, C. 1980, Scientific American 243:66, 70, incorporated herein by reference.

To prepare the inventive plasmids, the DNA therein is preferably ligated together to form a plasmid. For instance, the promoter, DNA encoding a fusion protein and antigen or epitopic DNA is preferably isolated, purified and ligated together in a 5′ to 3′ upstream to downstream orientation.

Accordingly, the inventive methods and products therefrom have several hereinstated utilities. Other utilities also exist for embodiments of the invention.

A better understanding of the present invention and of its many advantages will be had from the following examples given by way of illustration, and are not to be considered a limitation of the invention.

### EXAMPLES

### Example 1

Cloning and Expression of PspA

Using oligonucleotide primers LSM17 and LSM18, which were derived from the sequence of pspA from S. pneumoniae Rx1, polymerase chain reaction (PCR) was carried out on Rx1 genomic DNA by the method outlined in McDaniel et al. Microb. Pathogen. (1994), 17, 323-337. The sequence of LSM17 and LSM18 follow:

| | |
|---|---|
| LSM17: | 5′GCGGATCCGTAGCCAGTCAGTCTAAAGCTG3′ |
| LSM18: | 5′GCGGAATTCCCATTCACCATTGGCATTGACTTTAT3′ |

The amplified fragment of pspA (encoding full-length PspA), was cloned into pGT41. The plasmid pGT41 contains a CMV (HCMV-IE) promoter and a portion of the gene that encodes RSVG such that when an in-frame fusion is made, the resultant fusion protein may be transported to and anchored in the mammalian cell membrane where it can be exposed to the host immune system.

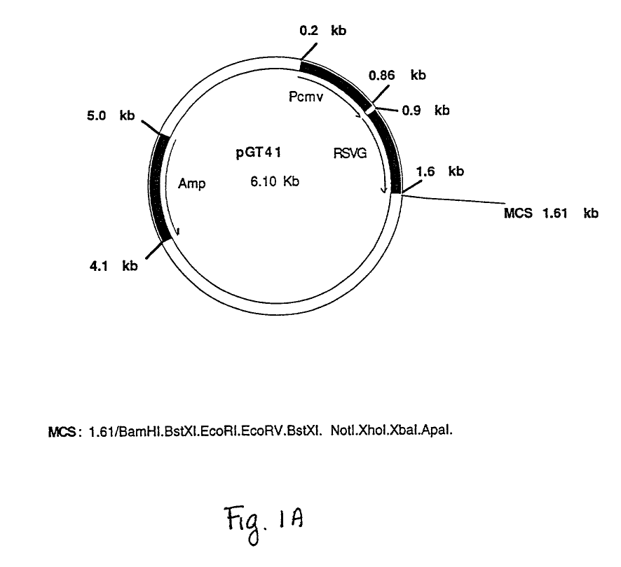

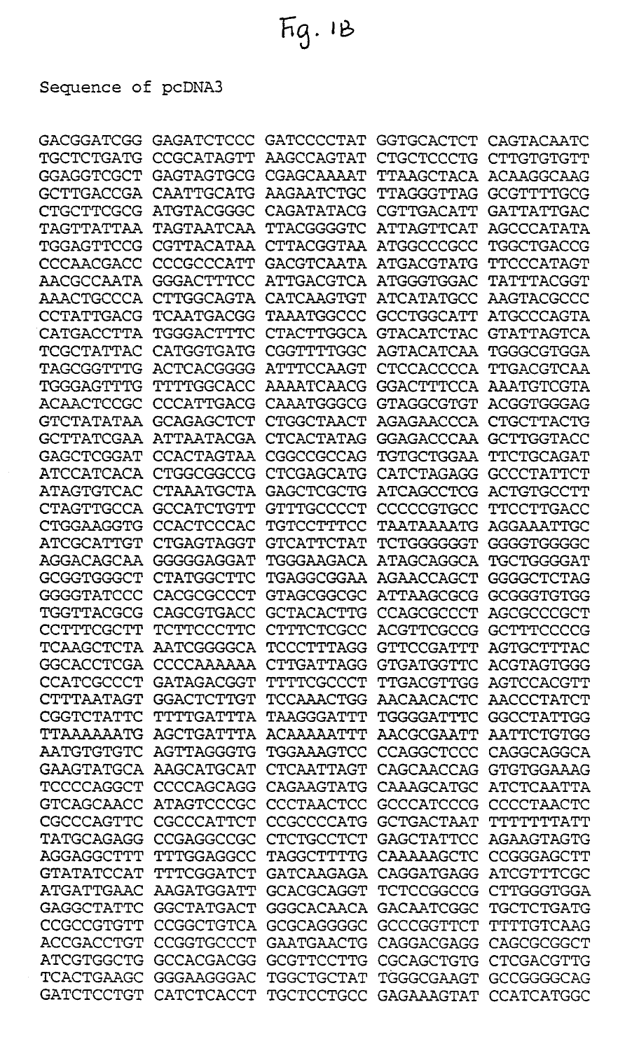

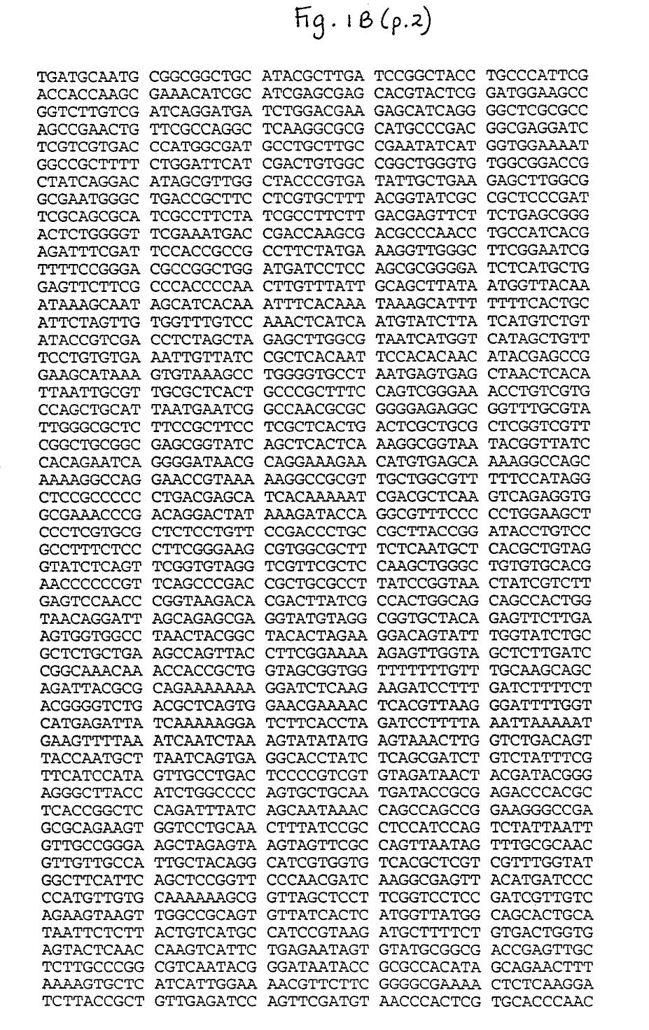

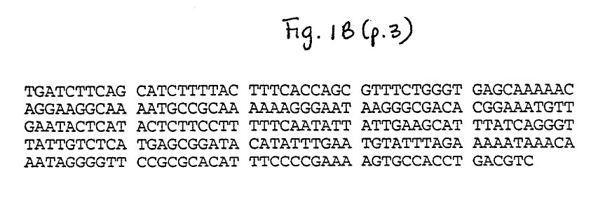

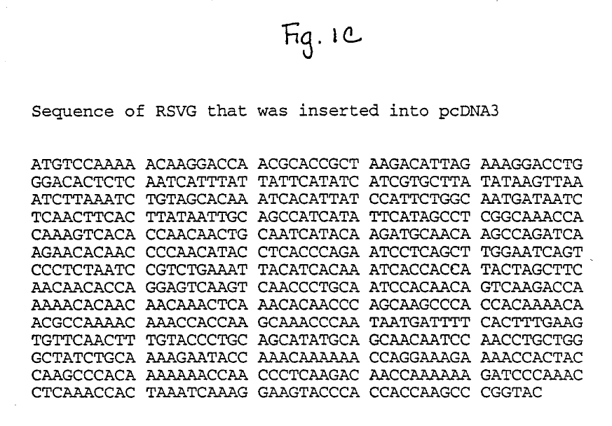

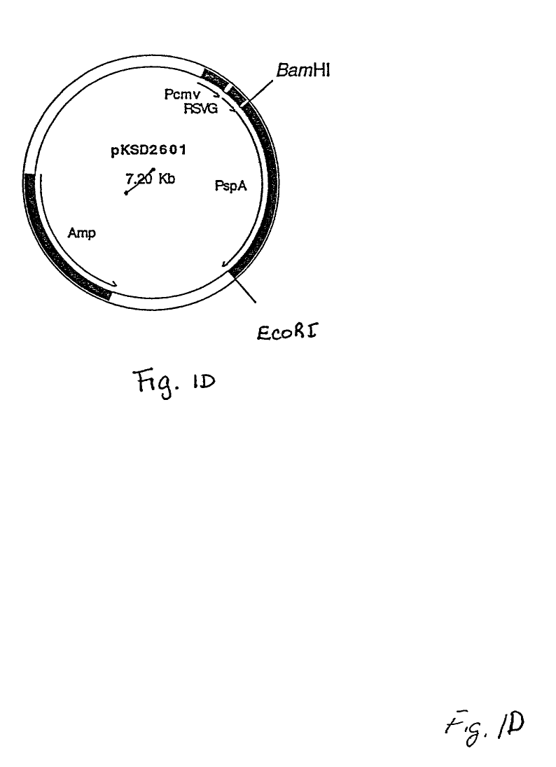

pGT41 was constructed using the commercially available plasmed pcDNA3 (Invitrogen). pcDNA3 was digested with KpnI, and a fragment of rsvG was amplified, digested wtih KpnI and ligated into the digested pcDNA3. The location of the rsvG was upstream of the multiple cloning site and downstream of the Pcmv. A diagram of pGT41 is shown in FIG. 1A, showing the salient features of the plasmed. The sequences of pcDNA3 and that of rsvG which was ligated into pcDNA3 to create pGT41 are shown in FIGS. 1B and 1C.

The plasmid derived from pGT41 containing the full-length pspA coding sequence was designated pKSD2601, shown in FIG. 1D. Sequencing confirmed the proper in-frame junction in pKSD2601. The pspA fragment amplified from genomic DNA of S. pneumoniae Rx1 using LSM17 and LSM18 was also digested with BamHI and EcoRI. The 5′ primer, LSM17, was designed such that when the amplified fragment was ligated into the BamHI-EcoRI site of pGT41, the resulting encoded protein would form a fusion between rsvG and PspA. pKSD2601 was constructed by digesting pGT41 with BamHI and EcoRI. These enzymes cut pGT41 within the ploy linker which is located down-stream of the CMV promotor and rsvG.

The plasmid pKSD2601 was used to transfect cultured HeLa cells to test for the expression of PspA in mammalian cells. The transfected cells were stained for both cytoplasmic and surface expression of PspA using anti-PspA monoclonal antibodies (MAbs) and a fluorescently labeled secondary antibody by the method outlined in McDaniel et al. Infection & Immunity (1988), 56, 3001-3003. PspA was expressed in the cytoplasm of the HeLa cells, but it was not present on the surface of the cells. In addition, only one of two MAbs, XiR278, was able to detect PspA. These findings could be indicative of a conformational change effecting the PspA epitope which is recognized by another MAb, Xi126.

### Example 2

Immunization with pKSD2601 Expressing PspA

pKSD2601 was used to immunize BALB/c mice. An additional group of mice received pGT41, the vector alone with no pneumococcal DNA inserted, as a control. Experiments were done twice using groups of five mice. Mice received lingual injections of 50 ug of purified plasmid at weekly intervals for five weeks. At the end of the sixth week, mice were bled and the PspA specific serum antibody level of each mouse was determined; the date is shown in Table 1. The antibody concentration was determined by an ELISA, in which the microtitration plates were coated with purified PspA versus control plates coated with purified PspA from the PspA- mutant pneumococcal strain WG44.1. An anti-PspA MAb of known concentration was used as a standard for estimation of antibody concentration in the mice.

TABLE 1
PspA specific antibody levels (ng/ml) in the serum of
BALB/c mice immunized with a plasmid (pKSD2601) expressing PspA

| | Immune | Control |
|---|---|---|
| Mouse Number | (ng/ml) | (ng/ml) |
| 1 | <13* | <13 |
| 2 | <13* | <13 |
| 3 | <13* | <13 |
| 4 | <13* | <13 |
| 5 | 80 | <13 |
| 6 | <13 | <13 |
| 7 | 272 | <13 |
| 8 | <13 | <13 |
| 9 | <13 | <13 |
| 10 | 96 | <13 |

*Indicates those mice that survived challenge with S. pneumoniae A66.

The saliva and feces were assayed for the presence of anti-PspA antibodies from a representative sample of the immunized and control mice. No anti-PspA antibodies were detected, which is indicative of a possible lack of a mucosal immune response.

The mice were then challenged intravenously with 2×10⁶ colony forming units (CFU) of capsular serotype 3 S. pneumoniae A66 (approximately 20×LD₅₀ for BALB/c mice). At 24 hours post challenge, the mice were bled by the method outlined in McDaniel et al. J. Immunol. (1984), 133, 3308-3312. The number of colony forming units of pneumococci per ml of blood was determined by plating 10 fold serial dilutions of the samples on blood agar. Although there was some overlap in the numbers of CFUs observed with the immunized and control mice, the immunized mice, on average, had about one and a half to greater than two times less pneumococci per ml of blood than the control mice on a logarithmic scale; the data is shown in Table 2. When the mean log CFU/ml for the two groups were compared (immunized=2.97±0.25 versus control=4.95±0.59), this difference was significant at p=0.0015 based on a Wilcoxin two sample rank test.

TABLE 2
Number (CFU/ML) of S. pneumoniae A66 in the blood of
BALB/c mice 24 Hrs. post challenge

| Mouse Number | Immune | Control |
|---|---|---|
| 1 | <93* | 2.05 × 10³ |
| 2 | <93* | 2.05 × 10³ |
| 3 | 1.86 × 10²* | 6.98 × 10³ |
| 4 | 2.65 × 10²* | 7.44 × 10³ |
| 5 | 6.51 × 10² | 3.95 × 10⁴ |
| 6 | 1.86 × 10³ | 5.14 × 10⁴ |
| 7 | 2.79 × 10³ | 6.53 × 10⁴ |
| 8 | 6.51 × 10³ | 7.00 × 10⁴ |
| 9 | 6.98 × 10³ | 4.17 × 10⁶ |
| 10 | 7.44 × 10³ | 3.49 × 10⁹ |

*Indicates the BALB/c mice that survived challenged with S. pneumoniae A66.

The effects of immunization could also be seen in protection from death, as shown in FIG. 2. Forty percent of the mice that were immunized with pKSD2601 survived, whereas none of the control mice survived. Moreover, the median time of death of the mice immunized with pKSD2601 was about 100 hours as compared to about 75 hours for the non-immune mice. The difference in survival time of the two groups was significant at p=0.007. These results indicate that intramuscular immunization with pKSD2601 plasmid DNA can induce protection against an otherwise lethal challenge with a capsular type 3 pneumococcus.

Furthermore, it was found that immunization with the DNA of a pneumococcal gene could elicit a protective immune response. Only 30 percent of the immunized mice had a detectable anti-PspA response, and this response did not correlate with the survival of the immunized mice, as shown in Table 1. One explanation for these findings is that the immunized mice that did survive challenge with the virulent pneumococci had anti-PspA antibodies that were not detectible in our anti-PspA assay. Alternatively, a cell mediated immune response to PspA may have contributed to the observed protection.

Accordingly, immunity or protection was afforded by the invention (with immunity or protection being understood to comprise the ability to resist or overcome infection or to overcome infection more easily than a subject not administered the invention, or to better tolerate infection than a subject not administered the invention, e.g., the present invention increases resistance to infection).

Having thus described in detail preferred embodiments of the present invention, it is to be understood that the invention defined by the appended claims is not to be limited by particular details set forth in the above description as many apparent variations thereof are possible without departing from the spirit or scope thereof.

### REFERENCES

1. Vogel, F. R. and N. Sarver (1995) Nucleic Acid Vaccines. Clin. Microbiol. Rev. 8: 406-410.

2. Whalen, R. G. (1996) DNA vaccines, cyberspace and self-help programs. Intervirology, In press.

3. Courvalin, P., Goussard, S., Grillot-Courvalen (1995) Gene transfer from bacteria to mammalian cells, C.R. Acad. Sci. Paris, Sciences de la vie, 318: 1207-1212.

4. Pardoll, D. M., A. M. Beckerleg, Exposing the immunology of naked DNA vaccines. Immunity, (1995) 3: 165-169.

5. Ulmer, J. B., Donnelly, J. J., Parker, S. E., Rhodes, G. H., Flegner, P. L., Dwarki, V. J., Gromkowski, S. H., Deck, R. R., DeWitt, C. M., Friedman, A., et al. Heterologous protection influenza by injection of DNA encoding a viral protein. Science 259, 1745-1749 (1993).

6. Ulmer, J .B., Deck, R. R., DeWitt, C. M., Friedman, A., Donnelly, J. J. & Liu, M. A. Protective immunity by intramuscular injection of low does of influenza virus DNA vaccines. Vaccine 12, 1541-1544 (1994).

7. McDaniel, L. S., Sheffield, J. S., Delucchi, P. & Briles, D. E. PspA, a surface protein of Streptococcus pneumoniae, is capable of eliciting protection against pneumococci of more than one capsular type. Infect Immun 59, 222-228 (1991).

8. Crain, M. J., Waltman, W. D., II, Turner, J. S., Yother, J., Talkington, D. E., McDaniel, L. M., Gray, B. M. & Briles, D. E. Pneumococcal surface protein A (PspA) is serologically highly variable and is expressed by all clinically important capsular serotypes of Streptococcus pneumoniae. Infect Immun 58, 3293-3299 (1990).

9. McDaniel, L. S., Yother, J., Vijayakumar, M., McGarry, L., Guild, W. R. & Briles, D. E. Use of insertional inactivation of facilitate studies of biological properties of pneumococcal surface protein A (PspA). J Exp Med 165, 381-394 (1987).

10. McDaniel, L. S., Scott, G., Widenhofer, K., Caroll, J. M. & Briles, D. E. Analysis of a surface protein of Streptococcus pneumoniae recognized by protective monoclonal antibodies. Micro Pathog 1, 519-531 (1986).

11. Waltman, W. D., II, McDaniel, L. S., Gray, B. M. & Briles, D. E. Variation in the molecular weight of PspA (Pneumococcal Surface Protein A) among Streptococcus pneumoniae. Micro Pathog 8, 61-69 (1990).

12. Tart, R. C., McDaniel, L. S., Ralph, B. A. & Briles, D .E. Truncated Streptococcus pneumoniae PspA molecules elicit cross-protective immunity againsty pneumococcal challenge in mice. J Inject Dis 173, 380-386 (1996).

13. Yother, J. & Briles, D. E. Structural properties and evolutionary relationships of PspA, a surface protein of Streptococcus pneumoniae, as revealed by sequence analysis. J Bact 174, 601-609 (1992).

14. McDaniel, L. S., Ralph, B. A., McDaniel, D. O. & Briles, D. E. Localization of protection-eliciting epitopes on PspA of Streptococcus pneumoniae between amino acids residues 192 and 260. Micro Pathogen 17, 323-337 (1994).

15. Yother, J. & White, J. M. Novel surface attachment mechanism of the Streptococcus pneumoniae protein PspA. J Bacteriol 176, 2976-2985 (1994).

16. McDaniel, L. S. & Briles, D. E. A Pneumococcal surface protein (PspB) that exhibits the same protease sensitivity as streptococcal R antigen. Infect and Immun 56, 3001-3003 (1988).

17. McDaniel, L. S., Benjamin, W. H., Jr., Forman, C. & Briles, D. E. Blood clearance by anti-phosphocholine antibodies as a mechanism of protection in experimental pneumococcal bacteremia. J. Immunol 133, 3308-3312 (1984).

What is claimed is:

1. A plasmid comprising DNA for expression of coding DNA by a eukayotic cell, wherein the coding DNA encodes a pneumococcal epitope of interest.

2. The plasmid of claim 1 wherein the DNA, from upstream to downstream, comprises: DNA encoding a promoter for driving expression in a eukaryotic cell, DNA encoding a leader sequence which facilitates expression, translation through or transport of the expression product in a eukaryotic cell membrane and DNA encoding a pneumococcal epitope of interest.

3. The plasmid of claim 2 wherein the promoter is a mammalian virus promoter.

4. The plasmid of claim 3 wherein the promoter is a cytomegalovirus promoter.

5. The plasmid of claim 2 wherein the DNA encoding a leader sequence is RSVG.

6. The plasmid of any one of claims 1 to 5 wherein the pneumococcal epitope of interest comprises a PspA, a fragment thereof, or a mixture thereof.

7. An immunological composition comprising a plasmid as claimed in any one of claims 1 to 5 and a carrier or diluent.

8. An immunological composition comprising a plasmid as claimed in claim 6 and a carrier or diluent.

9. A method for eliciting an immunological response in a host susceptible to pneumococcal infection, comprising administering to the host the composition as claimed in claim 7.

10. A method for eliciting an immunological response in a host susceptible to pneumococcal infection, comprising administering to the host the composition as claimed in claim 8.

11. A method for expressing a pneumococcal epitope of interest in vitro comprising transfecting a eukaryotic cell with a plasmid as claimed in any one of claims 1 to 5.

12. A method for expressing a pneumococcal epitope of interest in vitro comprising transfecting a eukaryotic cell with a plasmid as claimed in claim 6.

13. The method of claim 12 wherein the epitope of interest comprises PspA, a fragment thereof, or mixtures thereof.

14. A method for eliciting an immunological response in a host susceptible to sepsis, comprising administering to the host the composition as claimed in claim 7.

15. A method for eliciting an immunological response in a host susceptible to sepsis, comprising administering to the host the composition as claimed in claim 8.

16. A vaccine comprising a plasmid as claimed in any one of claims 1 to 5 and a carrier or diluent.

17. A vaccine comprising a plasmid as claimed in claim 6 and a carrier or diluent.

18. A vaccine as claimed in any one of claims 16 and 17, and a cytokine.

19. A vaccine as claimed in claim 18 wherein the cytokine is selected from the group consisting of IL-1, IL-2, IL-4, IFNγ, D71, and TNFα.

20. A vaccine as claimed in any one of claims 16 and 17, and DNA encoding a cytokine, wherein said DNA is within the inventive plasmid, either upstream or downstream from the pneumococcal DNA, or in a plasmid of its own.

21. A vaccine as claimed in claim 20 wherein the DNA encoding the cytokine is selected from the group consisting of IL-1, IL-2, IL-4, IFNγ, D71 and TNFα.

22. A vaccine as claimed in any one of claims 16 and 17, and a bacterial delivery system.

23. A vaccine as claimed in claim 22 wherein the bacteria is selected from the group consisting of Shigella flexnero and Escherichia coli.

24. The vaccine of claim 20 wherin said plasmid comprises, from upstream to downstream: DNA encoding a promoter for driving expression in a eukaryotic cell, DNA encoding a leader sequence for facilitating expression in a eukaryotic cell, and transport through the eukaryotic cell membrane, and DNA encoding a cytokine or epitope of interest thereof.
